(19) United States
(12) Patent Application Publication
(10) Pub. No.: US 2015/0274791 A1
(43) Pub. Date: Oct. 1, 2015

(54) USE OF NATURAL ANTIOXIDANTS DURING ENZYMATIC HYDROLYSIS OF AQUATIC PROTEIN TO OBTAIN HIGH QUALITY AQUATIC PROTEIN HYDROLYSATES

(71) Applicant: MATIS OHF., Reykjavik (IS)

(72) Inventors: Sigrun Mjoll Halldorsdottir, Hafnarfjordur (IS); Hordur Gudjon Kristinsson, Hafnarfjordur (IS); Rosa Jonsdottir, Reykjavik (IS)

(21) Appl. No.: 14/439,377

(22) PCT Filed: Oct. 29, 2013
(86) PCT No.: PCT/IS2013/050008

§ 371 Date: Apr. 29, 2015

(30) Foreign Application Priority Data
Oct. 29, 2012 (IS) 9011

(51) Int. Cl. C07K 14/46; A61Q 19/00; A23L 1/305; A61K 38/01; A23J 3/34; A61K 8/97; A61K 36/03

(52) U.S. Cl. CPC C07K 14/461; A61K 8/975; A61Q 19/00; A61K 36/03; A61K 38/014; A23J 3/342; A23L 1/3053; A61K 2800/10; A61K 2800/522

(57) ABSTRACT

The present invention relates to the use of natural antioxidants from marine algae extracts, such as seaweed extracts and preferably Fuscus vesiculosus extract, during enzymatic hydrolysis of aquatic protein from species such as fish, aquatic mammals, crustaceans and/or mollusks, to obtain high quality aquatic protein hydrolysates (APHs), having a biological activity of interest, for human consumption and cosmetics. The natural antioxidants can inhibit oxidation during hydrolysis, contribute to an increase in the bioactivity and decrease the bitter taste of the final product. The process can vary in starting material, pre-treatment, type and amount of enzyme, hydrolysis conditions, time, degree of hydrolysis and post-treatment. The invention also concerns food products, food supplements, pet food, animal feed, fish feed, fertilizer, pharmaceutical preparations, compositions, medicine and/or cosmetics comprising APHs according to the invention.

### FIELD OF INVENTION

The present invention I within the field of food processing and production of food materials, more particularly production of protein hydrolysates from protein sources such as fish. The invention relates to the use of certain natural antioxidants in particular marine algae extracts such as extracts from Fucus sp. or other seaweed species, during enzymatic hydrolysis of aquatic protein from species such as fish, aquatic mammals, crustaceans and/or mollusks, to obtain high quality aquatic protein hydrolysates (APHs).

### TECHNICAL BACKGROUND AND PRIOR ART

Peptides isolated from various aquatic raw materials have numerous health beneficial bioactivities making them a desirable ingredient in health foods (Kristinsson 2007). A major challenge to commercialize bioactive aquatic protein ingredients of high consistent quality is their very high oxidative instability. Oxidation in muscle foods leads to major quality deterioration, loss in nutritional value and strong off-odors and flavors (Ladikos and Lougovois 1990). The consumer awareness of natural bioactive ingredients has increased indicating a place for aquatic peptides on the market. A tremendous amount of utilizable waste material is left over after aquatic processing. Better utilization of these by-products will add significant value to the seafood industry and reduce environmental impact (FAO 2005). Using enzyme hydrolysis to extract proteins from poorly utilized materials has been identified as a major processing method to make better use of our seafood resources (Kristinsson 2007). Several companies are producing bioactive fish protein hydrolysates (FPHs), or fish peptides, for the food, feed and supplement market. However, a close analysis of these products has demonstrated that the products are of poor quality, despite selling for a premium. Methods for producing fish protein hydrolysates of improved quality with improved desirable organoleptic properties would be appreciated.

### SUMMARY OF INVENTION

The objective of this invention is to address the problem of oxidation during hydrolysis of aquatic protein and non-optimal taste, and to provide consumers with high quality consistent aquatic peptide products having positive health effects.

We have identified that oxidation products arisen during the hydrolysis can also have negative effect on the bioactivity. We have also identified that the use of certain natural antioxidants during the processing of enzymatically hydrolysed aquatic protein can address the problem. The natural antioxidants inhibit oxidation during hydrolysis, contribute to an increase in the bioactivity of the hydrolysates and/or protect them from losing their bioactivity caused by oxidation.

We have further identified that the use of certain natural antioxidants according to this invention during enzymatic hydrolysis of aquatic protein can not only inhibit oxidation and enhance bioactive properties but also decreases the bitter taste of APHs, which is a major problem in their commercialization. This is evidenced with sensory panel tests in the accompanying examples.

The present invention provides a process for producing high quality APHs and FPHs, which process comprises adding to the protein enzyme reaction mix a natural antioxidant, such as in particular a marine algae extracts, before or during the hydrolysis reaction. This results in improved hydrolysates with desirable organoleptic properties and enhanced bioactivities. The invention further provides aquatic protein hydrolysates produced with the process of the invention, and products comprising the hydrolysates, and uses thereof.

### BRIEF DESCRIPTION OF FIGURES

FIG. 1 illustrates lipid oxidation according to TBARS (μmol MDA/kg sample) formation of the different fish protein hydrolysates during hydrolysis; FPH-Hb (FPH containing hemoglobin (Hb)), FPH-Hb-Fv (FPH containing Hb and Fucus vesiculosus extract (Fv)) and FPH-Hb-AA (FPH containing Hb and Ascorbic acid (AA)), as a function of degree of hydrolysis (%).

FIG. 2 illustrates the antioxidative properties of FPH-Hb, FPH-Hb-Fv and FPH-Hb-AA as assessed by the ORAC method (TE=Trolox equivalence).

FIG. 3 illustrates the DPPH radical scavenging ability of FPH-Hb, FPH-Hb-Fv and FPH-Hb-AA.

FIG. 4 illustrates the reducing power ability (ascorbic acid equivalence) of FPH-Hb, FPH-Hb-Fv and FPH-Hb-AA.

FIG. 5 illustrates the ACE-inhibiting properties of FPH-Hb and FPH-Hb-Fv presented as the IC₅₀ value (mg/ml).

FIG. 6 illustrates secondary lipid oxidation, TBARS (μmol MDA/kg sample), of cod frame mince (starting material), FPH-Fv (FPH containing Fucus vesiculosus extract (Fv)), FPH, freeze dried FPH-Fv and freeze dried FPH.

FIG. 7 illustrates a radar plot of QDA odour and taste attributes (mean scores) of freeze dried FPH and freeze dried FPH-Fv. The number of stars indicates the significant difference level: one star (*) for 0.05, two (**) for 0.005 and three (***) for 0.001.

FIG. 8 illustrates the antioxidant activity of FPH-Fv, FPH, freeze dried FPH-Fv and freeze dried FPH as assessed by the ORAC method (TE=Trolox equivalence).

FIG. 9 illustrates the cellular antioxidant activity of FPH-Fv, FPH, freeze dried FPH-Fv and freeze dried FPH as assessed by HepG2 cell antioxidant assay.

### DETAILED DESCRIPTION

The process of auto-oxidation and development of rancidity in foods involves a free radical chain mechanism proceeding via initiation, propagation, and termination:

### Initiation: LH→L.

### Propagation: L.+O₂→LOO.

LOO+LH→LOOH+L.

### Termination: LOO.+LOO.→

LOO.+L.→non-radical products
L.+L.→(Shahidi 1997).

During oxidation highly unstable free radicals and hydroperoxides are formed that vandalize pigments, flavors, and vitamins. Compounds, such as ketones, aldehydes, alcohols, hydrocarbons, acids, and epoxides, are formed during the oxidation of unsaturated fatty acids (Khayat 1983). These compounds can bind to protein and form insoluble lipid-protein complexes. Thus lipid oxidation processes lead to discoloration, drip losses, texture changes, off-flavor development (Decker and Hultin 1992) and production of potentially toxic compounds (Xiong 2000). In order to measure the progress of lipid oxidation it is necessary to follow the transformation and/or formation of reactants, intermediates and products. Since many of these compounds are very unstable, and since they are differently affected by the presence of oxygen, pro-oxidants and antioxidants, it is recommended to monitor more than one stage of the oxidation process. Thus it is recommended that two or more methods be used to obtain a more complete understanding of lipid oxidation (Pike 2003). Many methods have been developed to measure the different compounds as they form or degrade during lipid oxidation for different food systems. In our studies we have primarily used the three following methods to measure the oxidation during hydrolysation of aquatic protein with good result: formation of lipid hydroperoxides (primary oxidation product), thiobarbituric acid reactive substances (TBARS) (secondary oxidation product) and sensory properties. TBARS has been found to be a very good indicator of lipid oxidation in seafood products and is often well correlated with sensory tests (Beltran and Moral 1990; Lubis and Buckle 1990; Simeonidou and others 1997).

Many marine species are rich in polyunsaturated fatty acids and pro-oxidants such as hemoglobin and iron. These muscle constituents interact before, during and after enzymatic hydrolysis processing and may be carried over into the final aquatic protein product (Kristinsson 2007). The reaction conditions during hydrolysis can have a major impact on oxidation. Hemoglobin, the most potent pro-oxidant in aquatic muscle, is highly pH and temperature sensitive in terms of its activity. At acidic pH, hemoglobin is highly pro-oxidative, while it is quite stable at high pH (Kristinsson and Hultin 2004). Aquatic muscle cells and proteins are also greatly affected by changes in environmental conditions such as pH, T and ionic strength and thus have different susceptibility to oxidation (Huss 1995). Hydrolysates are often obtained by enzymatic incubation at 50-65° C. due to the proteolytic activity of the enzyme. These harsh conditions can propagate oxidation during hydrolysis and lead to the formation of undesirable compounds. Therefore, careful selection of conditions for hydrolysis of aquatic protein is necessary in addition to antioxidant strategies in order to obtain high-quality APHs for human consumption. The optimal conditions chosen for hydrolysation must be a compromise of the nature of the enzyme (optimum conditions for proteolytic activity) and the raw material (composition and condition).

Although synthetic antioxidants have shown a great capability of inhibiting oxidation many processors and consumers have a negative view of their use and there is evidence that they can have a negative impact on health (Adegoko and others 1998). Natural antioxidants are more favorably accepted than synthetic antioxidants (Shi and others 2001) and their use has grown greatly in the past years while use of synthetic antioxidants is declining. Natural antioxidants include phenolic and polyphenolic compounds, chelators, antioxidant vitamins and enzymes, as well as carotenoids and carnosine. Valued scientific prospects such as EFSA and FAO have defined standards for classifying compounds as “natural antioxidant” to permit the usage of the term in a list of ingredients in foodstuffs and other substances. The mechanism by which these antioxidants are involved in the control of auto-oxidation and rancidity prevention differentiate (Shahidi 1997).

In general, antioxidants can be divided into two types, according to their mode of action either the initiation or the propagation of oxidation. Many antioxidants may also inhibit the decomposition of hydroperoxides and act as oxygen scavengers. Compounds that inhibit initiation or preventive antioxidants include metal inactivators or chelators, hydroperoxide destroyers and ultraviolet stabilizers. Metal chelators function by removing or chelating metal catalysts to change their redox potential and inhibit reaction i.e. production of alkoxyl radicals and peroxyl radicals. Hydroperoxide destroyers are mainly reducing agents that convert hydroperoxides into stable hydroxy products. Phenolic antioxidants react generally with peroxyl radicals and form stable products and can thus be considered propagation inhibitors. The phenol rings act as electron traps to scavenge peroxy, superoxy, superoxide-anions and hydroxyl radicals (Frankel 2007). Polyphenols from natural sources have been shown to be effective in reducing post-harvest spoilage in fish (Banerjee 2006).

Research on phenolic compounds extracted from various Icelandic marine algae, including the brown algae Fucus vesiculosus have shown very promising results in in vitro antioxidant activity studies. Moreover, they have shown a great potential to inhibit Hb-mediated lipid oxidation in washed fish model systems and in fish protein isolates during storage (Wang and others 2010).

Protein hydrolysates produced with certain natural antioxidants according to the invention possess desirable bioactivities and can be used in prevention and treatment of ailments such as high blood pressure, damage caused by reactive oxygen species, degenerative diseases, thrombosis and immune related problems and diseases. Such use is within the scope of this invention.

Ascorbic acid (vitamin C) is also a naturally occurring compound with multiple antioxidant activities e.g. electron donor, metal chelator and peroxy radical scavenger, that is commonly used as an antioxidant food additive (Frankel 2007). The antioxidant activity of natural antioxidants such as alfa-Tocopherol (vitamin E), Caffeic acid, Cinnamic acid, Courmaric acid, Carnosic acid, Carnosol, Epicatechin (flavan-3-ol), Ferulic acid, Flavone and Rosmarinic acid, is primarily based on their radical scavengers ability. Antioxidants also differ in their solubility. For example, alfa-Tocopherol, a common food antioxidant, is a non-polar molecule and will be partitioned into a non-polar environment, such as lipid membranes, exerting its activity there, whereas, ascorbic acid is a polar molecule and is active in the aqueous phase.

Antioxidants can also work better synergistically, where they regenerate each other and a combination of alfa-Tocopherol and ascorbic acid is often desired. In an embodiment of the invention, a combination of these two antioxidants is added as antioxidant to the reaction mixture.

The antioxidant activity of natural antioxidant is dependent on many factors: synergism and antagonism of antioxidant combinations, system-type, concentration and environmental conditions. Therefore, careful selection of antioxidants and their combinations and concentration, for usage during hydrolysis of seafood and related protein materials is critical for the production of high-quality food products with desirable organoleptic and bioactive properties. A fundamental problem in the enzymatic hydrolysis of proteins and proteinaceous material is the formation of a bitter flavor due to the formation of short peptide fragments.

The bitter taste is believed to be the result of cleavage of proteins at amino acids with hydrophobic side chains. A surprising effect of the present invention is that bitter taste is significantly reduced, as evidenced with sensory panel tests. The sensory panel test show a reduction in almost all tested negative sensory attributes, such as significant reduction in bitter taste, soap taste, fish oil taste, dried fish taste, earthy odour, and fermentation odour.

Our studies present the first rigorous tests on the effect of natural antioxidants on oxidation processes during hydrolysis of aquatic proteins. Results indicate that the use of certain natural antioxidants can address the problem of oxidation during hydrolysis by enhancing oxidative stability during the process. Various aquatic muscle model systems have been used to simulate the different raw materials that are used to produce APHs. The systems have contained different levels of added pro-oxidants (e.g. hemoglobin, iron etc.), different levels of lipids and subjected to various conditions of enzyme hydrolysis that led to products of high bioactivity. Natural antioxidants have been added to the systems prior to hydrolysis with excellent results. The antioxidants inhibit oxidation during hydrolysis of aquatic protein based raw materials, enhance bioactivity and surprisingly and significantly decrease the bitter taste of the APHs.

The invention concerns APHs produced with certain natural antioxidants, in particular marine algae extracts from algae species with high antioxidant activity. The APHs are characterized in that they are obtained by enzymatic hydrolysis of at least one source of protein preferably chosen from a seafood resource including but not limited to fish, aquatic mammals, crustaceans and molluscs. The source material may in some embodiments comprise fish or animal flesh (muscle tissue), whole animals, viscera, fish heads, skin, bones or skin and/or bones with flesh residue, any combinations of the above, or other typical leftover/byproduct material, such as any byproduct material from fish or other aquatic animal processing, e.g. shrimp or other seafood processing.

Various enzymes can be employed in the invention. In some embodiments the said enzymatic hydrolysis is carried out by means of one or more proteases selected from but not limited to proteases from marine species and Bacillus strains, Subtilisin, including Subtilisin from Bacillus licheniformis such as Alcalase® Food Grade, other commercial enzymes such as Protamex®, Flavourzyme® (Novozyme A/S, Denmark) (protease from Aspergillus oryzae) and Neutrase® (Novozymes A/S, Denmark) and Protease A “Amano” 2, Protease M “Amano” and Protease P “Amano” 6 (Amano Enzymes Inc., Nagoya, Japan), Pescalase® and Fromase™ from Gist Brocades (subsidiary of DMS, Herleen, the Netherlands), Promod 31™ from Biocatalysts (Biocatalysts, Cardiff, Wales, UK) and Maxatase™ from Genencor (Dupont Genencor Science).

As further described herein, the process and methods of the invention make use of certain natural antioxidants, such as in particular a marine algae extract from one or more antioxidant rich algae species. The algae species can in some embodiments be seaweed species such as red, green or brown seaweed species. Seaweed species used in the invention can be but are not limited to Fucus species, Ascophyllum species, Laminara species, Alaria species, Pelvetia species, Pyropia species, Caulerpa species, Durvillaea species, Ulva species, Porphyra species, and Sargassum species. Of particular interest are seaweed species including Fucus spiralis, Fucus vesiculosus, Fucus distichus, Fucus serratus, Fucus ceranoides, Fucus gardneri, Fucus evanescens, Furcellaria lumbricalis, Ascophyllum nodosum, Laminara hyperborea, Laminaria saxatilis, Laminaria digitata, Laminaria ochroleuca, Laminaria pallida, Laminaria setchellii, Lessonia flavicans, Lessonia nigrescens, Lessonia trabeculata, Lessoniopsis littoralis, Macrocystis integrifolia, Macrocystis pyrifera, Mastocarpus papillatus, Mastocarpus stellatus, Mazzaella splendens, Monostroma grevillei, Nemacystus decipiens, Nereocystis luetkeana, Osmundea pinnatifida, Palmaria hecatensis, Palmaria mollis, Palmaria palmata, Pelvetia canaliculata, Porphyra purpurea, Porphyra umbilicalis, Postelsia palmaeformis Pterocladia lucida, Pyropia columbina, Pyropia perforata, Pyropia tenera, Pyropia yezoensis, Rissoella verruculosa, Saccharina angustata, Saccharina dentigera, Saccharina groenlandica, Saccharina japonica, Saccharina latissima, Saccharina longicruris, Saccharina sessilis, Sargassum filipendula, Sargassum fusiforme, Sargassum muticum, Stephanocystis osmundacea, Ulva intestinalis, Undaria pinnatifida, Vertebrata lanosa, Alaria esculenta, Alaria marginata, Pelvetia canaliculata, Chondroacanthus canaliculatus, Chondracanthus chamissoi, Chondrus crispus, Caulerpa lentillifera, Caulerpa racemosa, Codium fragile, Costaria costata, Caulerpa sertularioides, Durvillaea antarctica, Durvillaea potatorum, Ecklonia cava, Ecklonia maxima, Ecklonia radiata, Egregia menziesii, Eisenia arborea, Eisenia bicyclis, Eualaria fistulsoa, Eucheuma denticulatum, Gigartina skottsbergii, Gelidiella acerosa, Gelidium corneum, Halopteris scoparia, Iridaea cordata, Jania rubens, and Kappaphycus alvarezii. A marine algae extract according to the invention can comprise essentially one of the above species or a combination of two or more of the mentioned species. The above species are commercially harvested and have been used in some products. Antioxidant activity can be readily ascertained by methods such as those described in the accompanying examples.

Extracts from one or more of the said seaweed species can be obtained by various extraction methods known to the skilled person, a suitable extraction method may be selected depending on the species of choice. Solvents such as ethanol can be used to extract contents with high activity, such as described in the Examples for a particular Fucus species; the exemplified method is as well applicable to other useful seaweed species. Other extraction methods may as well be employed, such as with other solvents including but not limited to other alcohols such as tert-butanol, isopropyl alcohol, n-propyl alcohol, acetone, ethers, hexane or other hydrocarbons, aqueous mixture of water and water miscible solvent (e.g. 70% acetone in water), and the like; and supercritical CO2 extraction. Some useful extraction methods are described by Wang etl al (2009) incorporated herein by reference. In some embodiments, crude extracts or water extracts of seaweeds are used in the APHs of the invention.

Further antioxidants may in some embodiments be added, such as any combinations of one or more of α-Tocopherol, Ascorbic acid, Caffeic acid, Cinnamic acid, Courmaric acid, Carnosic acid, Carnosol, Epicatechin (flavan-3-ol), Ferulic acid, Flavone, Phlorotannins and Rosmarinic acid.

The present invention concerns a method of enzymatically obtaining APHs/FPHs desirable organoleptic qualities, nutritional qualities and bioactive properties. The method according to the invention preferably comprises some or all of the below steps:

Grinding, shredding, mincing, or mechanically disintegrating in any other feasible way of at least one protein source that is preferably selected from fish, aquatic mammals, crustaceans and/or molluscs, in the presence of water, so as to obtain minced or ground pulp, which is retained for the subsequent process steps.

Suitable adjustment may be desired or necessary, depending on the choice of enzyme and antioxidants and the particular starting material, such adjustments can be but are not limited to;
adjustment of the material to a protein content in the range of 0.1% to 30% w/v (protein/water), such as in the range from 1-10%, and more preferably in the range of 1-5%;
adjustment of the pH of said material to a pH in the range of 1 to 14, such as preferably in the range pH 5-9, such as the range pH 6-8.5, such as about 6.5, about 7.0, about 7.5, about 8.0, or about 8.5;
adjustment of the said material to a convenient temperature at which the said enzyme composition does not become heat inactivated, in the range 0 to 80° C., and preferably a temperature at which the enzyme has optimal activity, such as e.g. in the range from 4-10° C. for cold-active enzymes, or in the range from about 20-50° C. for other enzymes, such as in the range 25-40° C., or the range 30-40° C., such as in the range of about 35-38° C., e.g. about 35° C., about 37° C., or about 38° C.

In the context of the invention, an effective amount of the selected antioxidant(s) is an amount when the relative amount of active substances significantly reduce oxidation and the damaging effect of the oxidation during hydrolysis and/or protects or contributes to an increase in the bioactivity, compared with a product that is processed with the exact same method without the addition of natural antioxidants according to the invention. Depending on the activity, the algae species of choice and selected extraction method, the amount of extract can in certain embodiments be in the range from about 0.01 g/L to about 25 g/L of protein-enzyme hydrolysis reaction mix, such as in the range from 0.1 g/L to about 10 g/L, or from about 0.5 to about 10 g/L such as from about 1 to 10 g/L, or from about 0.1 g/L to about 2.5 g/L, such as in the range of 0.1 to 1 g/L, e.g. about 0.1 g/L, about 0.25 g/L, about 0.5 g/L or about 1.0 g/L.

The amount of antioxidant extract can also be expressed in activity units, such as e.g. in units of Phloroglucinol equivalents (PGE); preferably the amount of antioxidant extract added to the hydrolysis reaction mixture is in the range of about 2.5 to about 100 g PGE/L, such as in the range of 5-100 g PGE/L, and more preferably in the range 5-50 g PGE/L, or from 10-50 g PGE/L, and more preferably in the range 10-25 g PGE/L, such as about 10 g PGE/L, about 12.5 g PGE/L, about 15 g PGE/L, or about 20 g PGE/L. Another useful unit to express antioxidant activity is GAE (gallic acid equivalent). Other useful units of antioxidant activity include Ascorbic acid equivalence (AAE). In useful embodiments, the amount of antioxidant added corresponds to in the range of about 5-25 AAE per g of protein/peptides in the protein-hydrolysis reaction mixture, such as in the range of about 5-10 AAE per g, and more preferably in the range of 5-10 AAE per g and more preferably above about 6 AAE per g.

Antioxidant activity can also be evaluated by assessing peroxyl radical scavenging activity, such as measured by ORAC-FL assay and reported in units of Trolox equivalents (TE/g extract). The APHs of the present invention preferably have TE values of above about 500 μmol TE/g protein, and more preferably above about 600 μmol TE/g and even more preferably above about 700 μmol TE/g, such as above about 800 μmol TE/g.

A suitable enzyme may be chosen from but is not limited to any one or more of the following: proteases from marine species, proteases from Bacillus strains, Alcalase® Food Grade, Protamex®, Flavourzyme®, Neutrase®, Protease A “Amano” 2, Protease M “Amano”, Protease P “Amano” 6, Pescalase®, Fromase™, Promod 31™ from and Maxatase™, preferably in the range of an enzyme/protein source ratio in the range from about 0.01 to 8% w/v of the said enzymes, so as to obtain a reaction mixture.

The enzymatic hydrolysis of the said protein source is generally executed for a time period in the range from about 0.1 to 48 hours with an effective amount of enzyme, or until the degree of hydrolysis (% DH) has reached a desired value where the final product possess a bioactivity of interest in significant intensities in the range from about 2 to 70% DH, and preferably a DH in the range 10 to 60%, more preferably in the range 10-50%, such as in the range 10-40%, or in the range of 20-40%.

Generally, stoppage of the enzymatic hydrolysis is suitably achieved by deactivation of the said enzyme. Deactivation of the enzyme may be achieved e.g. by raising the temperature of the said reaction mixture to a level not below 60° C., for a period in the range of 5 to 60 minutes, followed by cooling on ice, or by altering the pH such as by decreasing or increasing the pH to a pH at which the protease becomes inactivated, such as below pH 5 or above pH 8, depending on the particular enzyme being used.

Preferably, the produced hydrolyzed aquatic peptide fraction is separated from solid material, such as by concentration and/or drying (optional). Separation of the protein hydrolysate is in one embodiment performed by sedimentation. In another embodiment separation of the protein hydrolysate is performed by filtration to reduce solid matter. According to one embodiment of the invention, separation of the protein hydrolysate is performed by filtration using ultra filtration (UF) membranes, preferably with molecular weight cut-offs of including but not limited to 30, 10, 5, 3 and 1 kDa. In one embodiment, the separation of the protein hydrolysate is performed by centrifugation at a speed between 500 and 10000 G and separation of the precipitated residue is thus obtained, which residue can be discarded.

After separation a final product may be collected, that is an aquatic protein hydrolysate, having desirable bioactive properties. The obtained hydrolysate may if desired be dried, e.g. by lyophilisation, for convenient storage until further use.

Any suitable method may be applied in order to mince or grind the protein source as desired, such s but not limited to mechanical grinding, shearing, mincing or the like. According to one embodiment of the invention, the said grinding of the said protein source is carried out using by-products of the said aquatic species.

According to one embodiment of the invention, the method also comprises subjecting the starting material to protein isolation by extracting proteins of interest from the starting raw material, and subsequent hydrolysis of extracted proteins recovered in the process (prior to dewatering or after dewatering).

According to one embodiment of the invention, the said grinding of the said protein source is carried out using collagen or gelatin produced from the said aquatic species, meaning that the source material is rich in collagen and/or gelatin, or that the source material has been enriched for these materials. APHs of this type according to the invention can be advantageously used to improve gelatin and/or collagen products for incorporation into cosmetics, such as, but not limited to, creams, shampoos; food supplements and foods.

According to one embodiment of the invention, the hydrolysis with added antioxidants can be performed on a pre-hydrolyzed raw material.

According to one embodiment of the invention, the degree of hydrolysis is followed or measured in the final product.

According to one embodiment of the invention, an effective amount of one compound or combinations of two or more compounds that are defined as “natural antioxidants” by a valued scientific prospect such as EFSA and FAO, is used as a further natural antioxidant.

According to one embodiment of the invention, the said method also comprises concentrating, of the said hydrolysates obtained and optionally freezing it. According to one embodiment of the invention, the said method also comprises the drying of the said hydrolysates obtained.

According to one embodiment of the invention, the said method also comprises incorporation of stabilizers such as but not limited to antimicrobials in the said hydrolysates obtained. According to one embodiment of the invention, the said method also comprises a deodorization treatment of the said hydrolysates obtained. Deodorization treatment may in some embodiments comprise treatment with charcoal adsorbent or the like, or other deodorizing methods known to the skilled person.

The enzymatic hydrolysis of the starting material with added natural antioxidants of the aforementioned aquatic species according to the method according to the invention makes it possible to obtain an aquatic protein hydrolysate having advantageous organoleptic, nutritional and bioactive properties to the consumer. The enzymatic hydrolysis is performed by an enzyme and a natural antioxidant carefully selected to make it possible to obtain a protein hydrolysate having the aforementioned properties sought. The method, through the nature of the enzyme, the composition and nature of the starting material, the antioxidants (one or more), hydrolysis temperature and hydrolysis pH affects and enhances the organoleptic, nutritional and bioactive qualities of the hydrolysate obtained. This hydrolysate can then be incorporated in food products, food supplements, pet foods, animal feed, fish feed, fertilizer, pharmaceutical preparations, compositions, medicine and/or cosmetics. Thus the present invention concerns food products, food supplements, pet foods, animal feed, fish feed, fertilizer, pharmaceutical preparations, compositions, medicine and/or cosmetics comprising APHs produced with natural antioxidants according to the invention as described herein.

APHs produced according to one embodiment of the invention can be put dried into capsules and/or dried or as a liquid into foods to enhance health benefits of the resulting food products.

APHs according to the invention can possess various desired qualities. As is evident from the accompanying examples, the antioxidant activity not only affects the processing of the protein source material and enhances certain properties of the resulting peptides, but also the antioxidant activity that remains in the final product is a beneficial quality of the product, useful in many applications. Accordingly, the protein hydrolysates of the invention can possess antioxidant activity used to prevent or treat oxidative stress inside the body (by oral intake, such as a food or feed additive, or excipient in pharmaceutical or nutraceutical formulations) and on the skin (in topical products, both cosmetics and pharmaceuticals).

Surprisingly, it has been found that APHs of the present invention possess anti-hypertensive properties, including but not limited to ACE-inhibiting properties, as is evidenced in the Examples. Thus, APHs of the invention can be used as a high blood pressure-preventing or reducing agent.

Other useful indications are as well contemplated, such as based antithrombotic properties used to prevent or treat thrombosis, possess immunomodulatory ability used to prevent or treat ailments and illnesses related to the immune system, anti-diabetic activities to prevent or treat ailments and illnesses related to diabetes, anti-carcinogenic activities to prevent or treat ailments and illnesses related to cancer, and appetite enhancing or suppressing activities.

The nutraceutical or pharmaceutical formulations incorporating an APH processed according to the invention can comprise ingredients normally used in this type of formulation such as binders, flavorings, preservatives or colorings and, in the case of food supplements or medications, may be in the form of tablets, granules or capsules. Formulation according to the invention can also be in the form of suspension or syrups.

According to one embodiment of the invention, the APHs can be produced from for example fish gelatin and/or collagen to improve gelatin and/or collagen products for incorporation into cosmetics, such as, but not limited to, creams, shampoos, food supplements and foods.

APHs produced according to one embodiment of the invention can have antioxidant activity used to prevent or treat oxidative stress inside the body (consumption) and on the skin (cosmetics). APHs produced according to one embodiment of the invention can have anti-hypertensive properties, including but not limited to ACE-inhibiting properties used to prevent or treat high blood pressure. APHs produced according to one embodiment of the invention can have antithrombotic properties used to prevent or treat thrombosis. APHs produced according to one embodiment of the invention can produce APHs that have immunomodulatory ability used to prevent or treat ailments and illnesses related to the immune system. APHs produced according to one embodiment of the invention can produce APHs that have anti-diabetic activities to prevent or treat ailments and illnesses related to diabetes. APHs produced according to one embodiment of the invention can produce APHs that have anti-carcinogenic activities to prevent or treat ailments and illnesses related to cancer. APHs produced according to one embodiment of the invention can produce APHs that have appetite enhancing or suppressing activities. APHs produced according to one embodiment of the invention can possess any eligible bioactivity that has been identified in aquatic protein hydrolysates.

The features of the invention mentioned above as well as others, will emerge more clearly from a reading of the following description of an example embodiment, the said example being intended to be illustrative and non-limiting.

### EXAMPLES

### Example 1

### The Effect of Natural Antioxidants on Haemoglobin-Mediated Lipid Oxidation During Enzymatic Hydrolysis of Cod Protein

Materials

Fresh cod (Gadus morhua) fillets used for the preparation of the washed cod model were obtained iced from Marland Ltd. (Reykjavík, Iceland) within 24-48 h of the time of catch. The fillets were skinned and all dark muscle, blood spots and excess connective tissue were removed. The white muscle was minced in a grinder (plate hole diameter 4.5 mm). The enzyme Protease P “Amano” 6 was provided by Amano enzyme company, Japan. The brown algae (Phaeophyta) Fucus vesiculosus (Linnaeus) used for the preparation of seaweed extractions was collected in the Hvassahraun coastal area near Hafnarfjör [symbol] ur, in South-west Iceland in October 2008. The seaweeds were washed with clean seawater to remove epiphytes and sand attached to the surface and transported to the laboratory. The samples were carefully rinsed with tap water. Small pieces were cut and then freeze dried, pulverised into fine powder and stored in tightly sealed polystyrene containers at −20° C. prior to extraction. All spectrophotometric measurements were carried out by POLARstar OPTIMA, BMG Labtech, Offenburg, Germany.

Bleeding of Fish, Preparation of Haemolysate and Quantification of Haemoglobin

Farmed Arctic char (Salvelinus alpinus) was kindly provided by the Department of Aquaculture and Fish biology at Hólar University College in Iceland and was anesthetised in phenoxyethanol (0.5 g/I) for 3 min. The fish was held belly up and 1 ml of blood drawn from the caudal vein with a disposable syringe, preloaded with 1 ml of 150 mM NaCl and sodium heparin (30 units/nil).

Hemolysate was prepared within 24 h of blood collection according to the method of Richards and Hultin (2000). The heparinised blood was washed with four volumes of ice cold 1.7% NaCl in 1 mM Tris, pH 8.0. The plasma was removed by centrifugation at 700 g for 10 min at 4° C. The red blood cells were then washed three times with ten volumes of the same buffer and centrifuged between washes as before. The cells were lysed in three volumes of 1 mM Tris, pH 8.0, for 1 h. The stroma was removed by adding one-tenth volume of 1 M NaCl before final ultracentrifugation at 28000 g for 15 min at 4° C. All materials and samples were kept on ice during preparation. The hemolysate was stored at −80° C. until use. The concentration of Hb was determined by the HemoCue system of plasma/low Hb microcuvettes and photometer (Hemocue, Ängelholm, Sweden), using a method based on Vanzetti's reagent and spectrophotometric determination of azide-methaemoglobin complexes at 570 nm (Jónsdóttir and others 2007). A standard curve with serial bovine Hb solution (ranging from 0-70 μmol/l) was used for calibration. Samples and standards were diluted with 50 mM Tris buffer (pH 8.6).

Preparation of Fucus vesiculosus EtOAc Fraction

The solvent extracts were prepared according to the method described by Wang and others (2009). Briefly, 40 g of dried algal powder were extracted with 200 ml 80% EtOH in a platform shaker for 24 h at 200 rpm and at room temperature. The mixture was centrifuged at 2500 g for 10 min at 4° C. and filtered. The filtrate was concentrated in vacua to a small volume and the residue was suspended in a mixture of methanol (MeOH) and water (40:30, v/v) and partitioned three times with n-hexane, EtOAc and n-butanol successively. The EtOAc soluble fraction was obtained after removal of solvent, and freeze-dried. The F. vesiculosus extract (F. vesiculosus EtOAc fraction) was stored in air tight containers at −20° C. until further use.

Total Phlorotannin Content

The total phlorotannin content (TPC) of the EtOAc F. vesiculosus fraction was determined by the Folin-Ciocalteu method described by Koivikko and others (2005). Results were expressed as mg gallic acid equivalent (GAE) per 100 g of extract.

Preparation of Washed Cod Muscle, WCM

Minced cod muscle was washed based on the method of Richards and Hultin (2000). All materials and samples were kept on ice during preparation. The mince was washed twice with Milli-Q water (1:3, w/w) and once with 50 mM sodium phosphate buffer (1:3, w/w pH 6.3). The washed mince was immediately frozen and kept at −80° C. until use.

Preparation of Fish Protein Hydrolysates (FPH)

The WCM was thawed under cold running tap water, diluted in water to 3% protein and adjusted to pH 8 with 1 M NaOH. Different combinations of Hb, L-ascorbic acid and F. vesiculosus extract were added to the system (Table 1). Protease P “Amano” 6 was used to hydrolyze the different variations of system at pH 8 and 36° C. to achieve 20% degrees of hydrolysis (% DH).

TABLE 1
Detailed information on samples

| Sample | Ingredients |
|---|---|
| FPH | FPH prepared from WCM system (3% protein at pH 8) |
| FPH-Hb | FPH prepared from WCM system (3% protein at pH 8) with |
| | added Hb (20 μM/kg) |
| FPH-Hb- | FPH prepared from WCM system (3% protein at pH 8) with |
| Fv | added Hb (20 μM/kg) and F. vesiculosus EtOAc fraction^{a} |
| | (200 ppm) |
| FPH-Hb- | FPH prepared from WCM system (3% protein at pH 8) with |
| AA | added Hb (20 μM/kg) and L-ascorbic acid (200 ppm) |
| Control | Unhydrolysed WCM system (3% protein at pH 8) |

^{a}Total phlorotannin content: 62.0 ± 6.1 g PGE/100 g.

The % DH with time was monitored with reference to equation 1 (Adler-Nissen 1986):

% DH=B×N_{base}/α×hₜₒₜₐₗ×MP×100 (1)

where B=volume of base used, N_{base}=normality of base, α=degree of dissociation, hₜₒₜₐₗ=total number of peptide bonds per mass unit and MP=amount of protein used. The degree of dissociation (α) was found by equation 2:

α=10^{pH-pKa}/1+10^{pH-pKa} (2)

here pH is the value at which the enzyme hydrolysis was performed. The pKa values were calculated according to equation 3 (Steinhardt and Beychok 1964):

pKa=7.8+((298−T)/298×T)×2400 (3)

where T is the temperature in Kelvin at which the enzyme hydrolysis was performed.

Samples were taken periodically during the hydrolysis (at 0, 4, 8, 12, 16 and 20% DH) and once 20% DH was achieved, the solution was placed in a zip-locked bag to increase the surface area, and heated to 90° C. for 10 min to inactivate the enzymes, followed by cooling on ice. Hydrolyzed samples were stored at −20° C. until further use. Protein content was measured in the solutions by the Dumas method using a macro analyzer vario MAX CN equipment (Elementar Analysensysteme GmbH, Germany). A factor of 6.25 was used to convert nitrogen to crude protein content.

Lipid Hydroperoxides

Lipid hydroperoxides were determined with a modified version of the ferric thiocyanate method (Santha and Decker 1994). Total lipids were extracted from the hydrolysates with 1 ml ice-cold chloroform:MeOH (1:1, v/v) solution, containing 500 ppm butylated hydroxytoluene (BHT) in the ratio (2:1, v/v). Sodium chloride (0.5 M) was added (250 μl) to the mixture and vortexed for 30 s before centrifuging at 2350 g for 5 min (Model Z323K, Hermle laboratories, Germany). The chloroform layer was collected (400 μl) and completed with 600 μl of ice-cold chloroform:MeOH solution. A total of 5 μl ammonium thiocyanate (4M) and ferrous chloride (8 mM) were finally added. The samples were incubated at room temperature for 10 min and read at 500 nm in the POLARstar OPTIMA. A standard curve was prepared using cumene hydroperoxides. The results were expressed as mmol lipid hydroperoxides per kilogram of sample.

Thiobarbituric Acid-Reactive Substances (TBARS)

A modified method of Lemon (1975) was used for measuring TBARS. A sample (0.1 ml) was vortexed with 0.6 mL of trichloroacetic acid (TCA) extraction solution (7.5% TCA, 0.1% propyl gallate and 0.1% ethylenediaminetetraacetic acid mixture prepared in ultra-pure water) for 10 seconds. The homogenized samples were completed with 0.4 mL TCA extraction solution and centrifuged at 9400 g for 15 min. The supernatant (0.5 ml) was collected and mixed with the same volume (0.5 ml) of thiobarbituric acid (0.02 M) and heated in a water bath at 95° C. for 40 min. The samples were cooled down on ice and immediately loaded into 96-wells microplates (NUNC A/S Thermo Fisher Scientific, Roskilde, Denmark) for reading at 530 nm in the POLARstar Optima. A standard curve was prepared using tetraethoxypropane. The results were expressed as μmol of malonaldehyde diethylacetal (MDA) per kg of sample.

Oxygen Radical Absorbance Capacity (ORAC)

The oxygen radical absorbance capacity (ORAC) assay was performed according to Ganske and Dell (2006) with slight modifications. Different dilutions of Trolox (3.125-50 μM) and samples were prepared in phosphate buffer (10 mM, pH 7.4). Into every working well of a black opaque microplate (200 μl, 96 wells, MJ Research, USA) the following was pipetted in triplicate: 1) 60 μL of 10 nM Fluorescein solution; 2) 10 μL of Trolox dilutions for standard; 10 μL of sample solution; 10 μL of phosphate buffer for blank.

The microplate was incubated for 15 min at 37° C. without shaking in the POLARstar Optima. After incubation, 30 μL of 120 mM AAPH solution were quickly added manually using a multi-channel pipette. The fluorescence was recorded every 0.5 min for the first 40 cycles and every min for the last 60 cycles. The filters used for excitation were 485 nm and 520 nm for emission. The total time for the measurement was 80 min.

The antioxidant curves (fluorescence versus time) were normalized. The data from the curves were multiplied by the factor:

$$\frac{{fluorescence}_{blank , t = 0}}{{fluorescence}_{sample , t = 0}} \qquad ( 5 )$$

The area under the fluorescence decay curve (AUC) was calculated by the normalized curves using the following equation:

AUC=(0.5+f0.5/f0+ . . . +f19,5/f0)×0.5+(f21/f0+ . . . +f78/f0)+0.75 f20/f0+0.5 f79/f0 (4)

where f0 was the fluorescence reading at the initiation of the reaction and f79 was the last measurement.

The net AUC was obtained by subtracting the AUC of the blank from that of a sample or standard. The ORAC value was calculated and expressed as micromoles of Trolox equivalents per gram of protein (μmol of TE/g protein) using the calibration curve of Trolox.

Metal Chelating Ability

The metal chelating ability of the hydrolysates was evaluated at 0.15% protein concentration using the method of Boyer and McCleary (1987) with a slight modification. The following solutions were prepared for this assay: 2 mM ferrous chloride (FeCl₂) and 5 mM 3-(2-pyridyl)-5,6-bis(4-phenyl-sulfonic acid)-1,2,4-triazine (ferrozine). Sample solutions (150 μl) were mixed with 5 μl of 2 mM FeCl₂ in a microplate. Distilled water (150 μl) was used instead of sample solution as a blank. The reaction was initiated by the addition of 10 μl of 5 mM ferrozine. Distilled water (10 μl) instead of ferrozine was used in the control. The solutions were well mixed and allowed to stand for 30 min at room temperature. After incubation, the colour change resulting from metal chelating was measured spectrophotometrically at 560 nm in the POLARstar Optima. The results were calculated according to the following formula:

Chelating activity (%)=A_{blank}(Aₛₐₘₚₗₑ−A_{control})A_{blank}×100 (6)

where A_{blank}=absorbance of the blank, Aₛₐₘₚₗₑ=absorbance of the sample, A_{control}=absorbance of the control samples at 560 nm.

2,2-diphenyl-1-picryhydrazyl (DPPH) Radical Scavenging Activity

DPPH radical scavenging activity was determined as described by Wu and others (2003) with a slight modification. Protein sample solutions (1.5 mg/ml) was diluted in 95% methanol (1:9, v/v) and centrifuged at 10000×g for 10 min, 150 μl of the supernatant was collected and mixed with 60 μl of DPPH in 0.02% MeOH solution, 150 μl of water instead of supernatant was used as blank, and 60 μl of MeOH was used instead of DPPH solution as control. This procedure was carried out on a microplate and allowed to stand at room temperature in the dark for 30 min. The absorbance of the resultant solution was read at 520 nm. The scavenging effect was calculated as follows:

$$\% inhibition = \frac{Ablank - ( Asample - Acontrol )}{Ablank} \times 100 \qquad ( 7 )$$

where A_{blank} is the absorbance of the blank, Aₛₐₘₚₗₑ is the absorbance of the sample and A_{control} is the absorbance of the control at 520 nm in the POLARstar Optima.

Reducing Power

The reducing power of the hydrolysates was measured using a modified method of Benjakul and others (2005) at 0.15% protein concentrations. In brief, the method involves mixing 50 μL of protein samples or distilled water (control) with 250 μl of 0.2 M phosphate buffer (pH 6.6) and 250 μl of 1% potassium ferricyanide solution. The mixture was digested at 50° C. for 30 min, then mixed with 250 μL of 10% TCA solution and centrifuged at 8161.2 g for 10 min. Two hundred μL of the supernatant were collected and mixed with 40 μl of 0.1% ferric chloride (FeCl₃) solution. After 10 min of incubation at room temperature, while shaking, the absorbance of the supernatant was read at 700 nm in the POLARstar Optima. The relative activity of the sample was calculated in relation to the activity of ascorbic acid standards (0-200 μg/ml) and the results were expressed as mg of ascorbic acid equivalents per g of protein. Increased absorbance of the reaction mixture indicates the increasing reducing power.

Angiotensin Converting Enzyme (ACE) Inhibitor Activity

ACE activity was measured according to Vermeirssen and others (2003) with some modifications. Distilled water (blank) or inhibitor solution (20 μl) was mixed with 10 μl of 0.2 U/ml angiotensin I converting enzyme from rabbit lung (Sigma-Aldrich, St. Louis, Mo.) and the mixture solution was pre-incubated at 37° C. for 15 min in a microplate. Subsequently 170 μl of the substrate solution (0.5 mM N-[3-(2-Furyl)acryloyl]-Phe-Gly-Gly in 50 mM Tris-HCl buffer containing 300 mM NaCl at pH 7.5) were added manually with a microchannel pipette. The microplates were quickly placed in a POLARstar Optima microplate reader that was set at 37° C. and recorded every minute at 340 nm in the POLARstar Optima for 60 min. The ACE inhibitor activity (%) was calculated as:

ACE inhibitor activity (%)=1−(Δₛₐₘₚₗₑ/Δ_{control})×100 (8)

where Δₛₐₘₚₗₑ is Δ_{control} is the slope of the sample with hydrolysates, and needed the slope of the control sample. The concentration of selected protein hydrolysates needed to inhibit the ACE by 50% (IC₅₀) was determined by assaying hydrolysate samples at different protein concentrations and plotting the ACE inhibition percentage as a function of protein concentration.

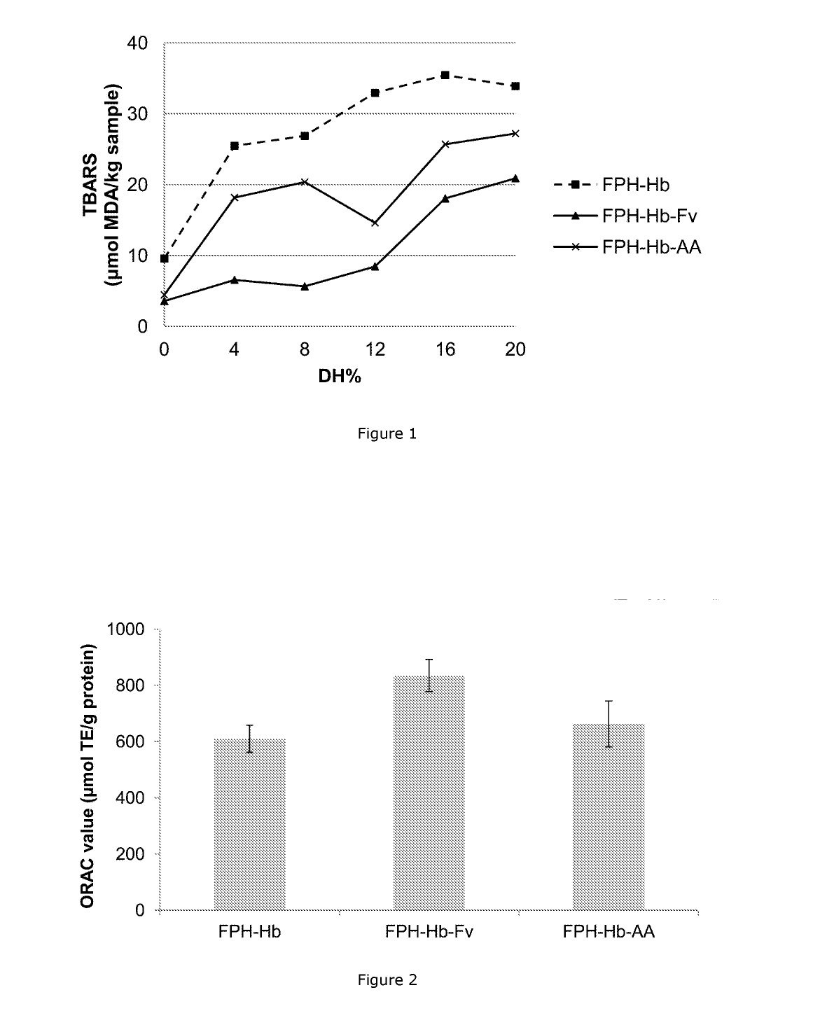

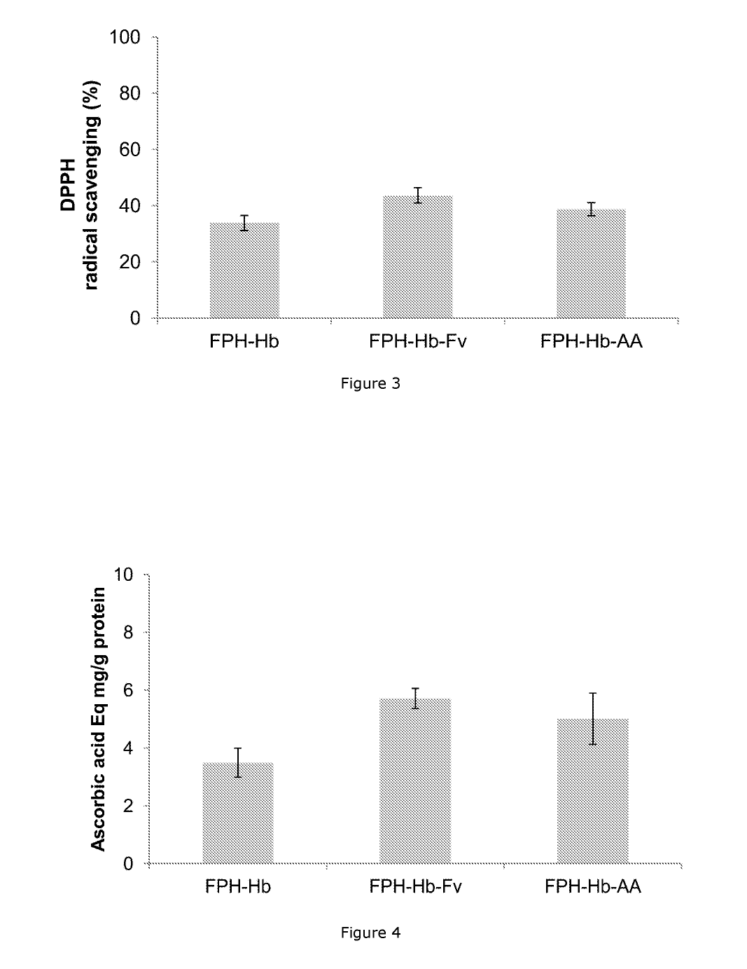

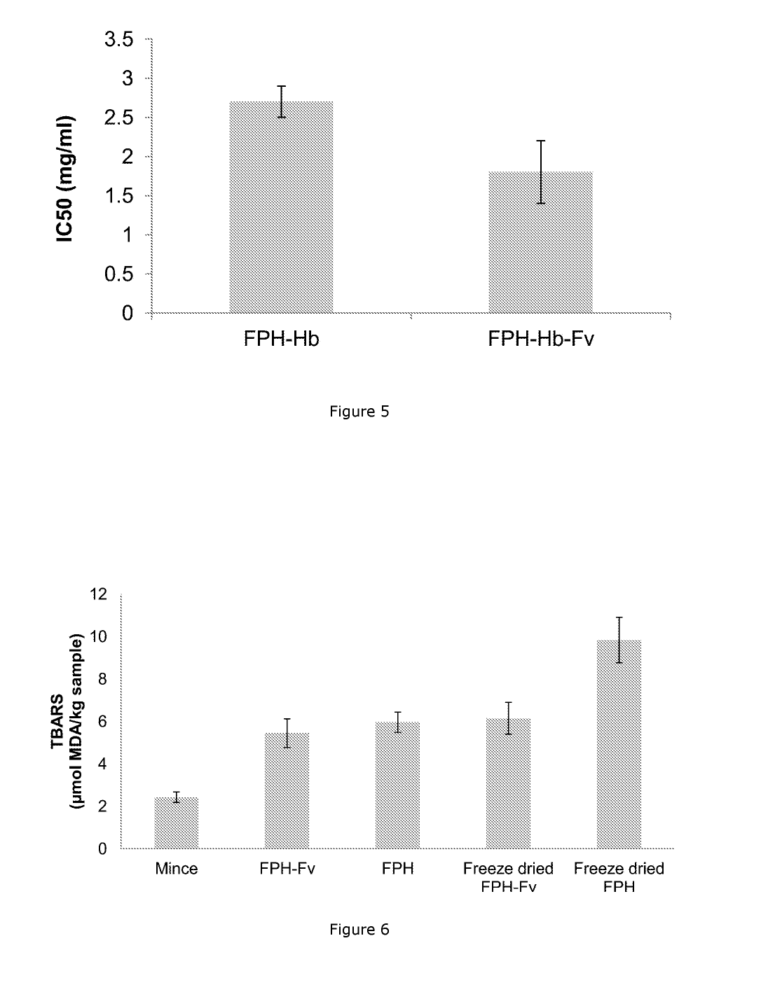

TBARS measurements show that the antioxidants, Fucus vesiculosus extract and L-ascorbic acid, significantly reduced oxidation during hydrolysis of cod protein with added hemoglobin (FIG. 1). A significant increase in antioxidant activity was observed in samples with added natural antioxidants (FPH-Hb-AA and FPH-Hb-Fv) (FIGS. 2-4). A significant increase in ACE-inhibiting properties was observed in samples with added Fucus vesiculosus extract (FPH-Hb-Fv) (FIG. 5).

### Example 2

### Effect of Fucus vesiculosus Extract on Lipid Oxidation During Hydrolysis of Fish Protein

MPF Island ehf. (Grindavik) provided cod mince from byproducts (cod frames) produced in January 2011. The cod mince was diluted in water to 3.7% protein and adjusted to pH 8 with 2 M NaOH. It was divided in to two 1 L portions, one containing 240 ppm seaweed extract (52.9 PGE/100 g extract) (FPH+seaweed) and the other not (FPH). The material was subjected to enzyme (protease P “Amano” 6) for hydrolysis to achieve 20% degrees of hydrolysis (% DH). The samples were freeze dried and stored in −20° C. until analyzed. Enzymatic hydrolysation, preparation of Fucus vesiculosus EtOAc fraction, measurement of total phlorotannin content, lipid hydroperoxides, TBARS and ORAC analysis were according to the methods described in Example 1.

Sensory Evaluation

Protein solutions were prepared from FPH and FPH+seaweed. 15 g of freeze dried protein powder was mixed with distilled water up to 250 mL. The two mixtures of protein solutions were evaluated with QDA (Quantitative Descriptive Analysis), introduced by Stone and Sidel (1985).

TABLE 2
The sensory attributes.
ODOR

| | |
|---|---|
| Fresh fish | Odor of fresh raw fish fillet |
| Sweet | Sweet odor, fresh fruit e.g. pear and melon |
| Earthy | Earthy odor, clay, humus |
| Sour | Sour odor, whey |
| Fermentation | Fermentation odor |
| TMA | TMA odor, trimethylamine |
| Rancid | Rancid odor |

FLAVOR

| | |
|---|---|
| Bitter | Bitter or pungent flavor |
| Sour | Sour basic flavor |
| Soap | Soapy or chemical flavor |
| Fish oil | Flavor of fish oil |
| Rancid | Rancid flavor |
| Dried fish | Flavor of dried fish, trimethylamine |

Eight panelists, all trained according to international standards (ISO 1993), participated in the sensory evaluation. The panelists were familiar with the QDA method and experienced in sensory analysis of protein solutions. One training session was used to synchronize the panel prior to the sensory evaluation. In the training session the panelists were trained in recognition of sensory characteristics of the samples and describing the intensity of each attribute for a given sample using an end anchored linear scale. In addition, the sensory attributes of the samples were defined using a QDA scale from an earlier experiment as a reference. The sensory attributes were thirteen, describing the odor and flavor of the samples (table 2). Each sample was 6 mL of protein solution presented in a small plastic beaker. The samples were coded with three digit numbers and presented according to latine square method. The sensory evaluation was carried out in one session with a duplicate from both sample groups. A computerized system (FIZZ, Version 2.0, 1994-2000, Biosystémes) was used for data recording.

Intracellular Antioxidative Activity by HepG2 Cell Assay

An intracellular antioxidant assay was performed on FPH and FPH+seaweed, using HepG2 cells maintained in Minimum Essential Medium α (MEMα), supplemented with 10% (v/v) heat-inactivated fetal bovine serum, penicillin (50 units/mL), and streptomycin (50 μg/mL). Cells were incubated at 37° C. in a fully humidified environment under 5% CO₂, and HepG2 cells at passage 80-100 were used for the experiments. Cells were subcultured at 3-5 days intervals before reaching 90% confluence.

The assay was done using HepG2 cells at a density of 6×10⁴/well using black 96-well plates (BD Falcon™) in 100 μL growth medium/well according to Wolfe and Liu (2007) and Samaranayaka and others (2010) with minor modifications. Twenty four hours after seeding, 100 μL of DCFH-DA probe (1 μM in HBSS) was added to the cells and incubated at 37° C. in the dark for 30 min. Cells were then treated with different concentrations of FPH and FPH+seaweed, and incubated for 1 h at 37° C. This was followed by the addition of 100 μL of AAPH peroxyl radical initiator (final concentration 500 μM AAPH in HBSS) to the cultured cells after removal of the test compounds. Fluorescence readings (λ_{excitation}=493 nm, λₑₘᵢₛₛᵢₒₙ=527 nm) were recorded using a POLARstar OPTIMA (BMG Labtech) every 10 min for 90 min after addition of AAPH. Each plate included four replicates of control and blank wells: Blank wells contained cells exposed only to the DCFH-DA probe. The control consisted of cells with DCFH-DA probe and the AAPH added but in the absence of test compounds.

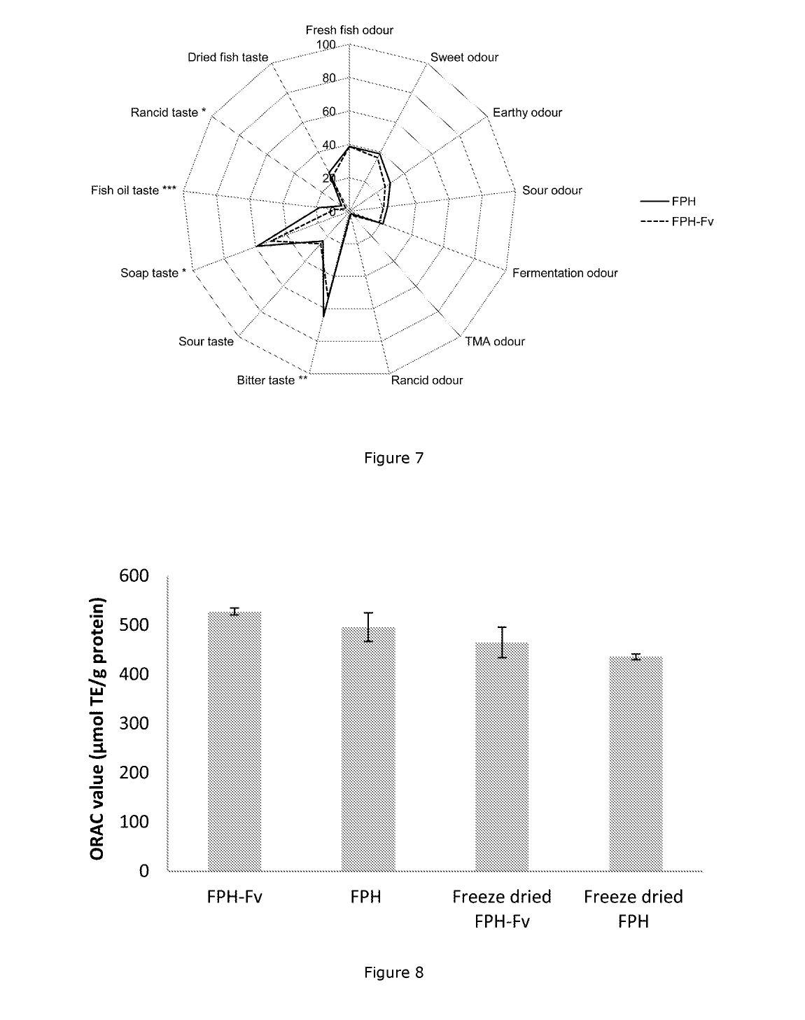

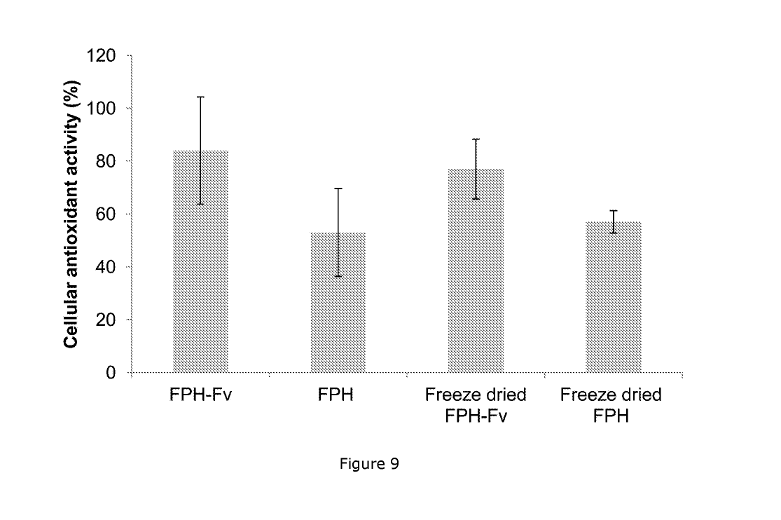

TBARS measurements show that the Fucus vesiculosus extract significantly reduced oxidation in during hydrolysis and freeze drying of cod frame mince (FIG. 6). The FPH containing Fucus vesiculosus extract (FPH-Fv) had significantly less bitter, soap, fish oil and rancitity taste than the FPH not containing any antioxidants (FPH) (FIG. 7). A significant increase in antioxidant activity was observed in samples containing Fucus vesiculosus extract (FPH-Fv) according to both the ORAC method and the HepG2 cell antioxidant assay (FIGS. 8-9).

### REFERENCES

1. Adegoko G O, Vijay Kumar M, Gopala Krishna A G, Varadaraj M C, Sambaiah K, Lokesh B R. 1998. Antioxidants and lipid oxidation in foods: A critical appraisal. Journal of food science and technology 35:283-98.
2. Adler-Nissen J. 1986. Enzymatic hydrolysis of food proteins. London, England: Elsevier Applied Science Publishers Ltd.
3. Banerjee S. 2006. Inhibition of mackerel (Scomber scombrus) muscle lipoxygenase by green tea polyphenols. Food Res Int 39:486-91.
4. Beltran A, Moral A. 1990. Gas chromatographic estimation of oxidative deterioration in sardine during frozen storage. Lebensm Wiss Technol Food Sci Technol 23:499-504.
5. Benjakul S, Visessanguan W, Phongkanpai V, Tanaka M. 2005. Antioxidative activity of caramelisation products and their preventive effect on lipid oxidation in fish mince. Food Chem 90:231-9.
6. Boyer R F, McCleary J C. 1987. Superoxide ion as a primary reductant in ascorbate-mediated ferretin iron release. Free Radical Bio Med 3:389-95.
7. Decker E, Hultin H. 1992. Lipid Oxidation in Muscle Foods via Redox Iron. Lipid Oxidation in Food. American Chemical Society:33-54.
8. FAO. 2005. Review of the state of world marine fishery resources. FAO Fisheries Technical Paper. p. 457.
9. Frankel E N. 2007. Antioxidants in Food and Biology—Facts and Fiction. Bridgwater: The Oily Press, (Chapter 4).
10. Ganske F, Dell E J. 2006. Assay on the FLUOstar OPTIMA to Determine Antioxidant Capacity. Offenburg: BMG LABTECH. Application Note 148. Rev. 12/2006
11. Huss H. 1995. Quality and quality changes in fresh fish. FAO Fisheries Technical Paper 348. Rome: FAO.
12. Jónsdóttir R, Bragadóttir M, Ólafsdóttir G. 2007. The Role of Volatile Compounds in Odor Development During Hemoglobin-Mediated Oxidation of Cod Muscle Membrane Lipids. J Aquat Food Prod Tech 16:67-86.
13. Khayat A S, D. 1983. Lipid oxidation in seafood. Food Technology 7:130-40.
14. Koivikko R, Loponen J, Honkanen T, Jormalainen V. 2005. Contents of soluble, cell-wall-bound and exuded phlorotannins in the brown alga Fucus vesiculosus, with implications on their ecological functions. J Chem Ecol 31:195-212.
15. Kristinsson H G. 2007. Aquatic food protein hydrolysates. In: Shahidi F, editor. Maximising the value of marine by-products. Cambridge England: Woodhead Publishing. p 229-47.
16. Kristinsson H G, Hultin H O. 2004. The Effect of Acid and Alkali Unfolding and Subsequent Refolding on the Pro-oxidative Activity of Trout Hemoglobin. Journal of Agricultural and Food Chemistry 52:5482-90.
17. Ladikos D, Lougovois V. 1990. Lipid oxidation in muscle foods: A review. Food Chemistry 35:295-314.
18. Lemon D W. 1975. An improved TBA test for rancidity. New series circular 51. Halifax Laboratory, Halifax, Nova Scotia.
19. Lubis Z, Buckle K A. 1990. Rancidity and lipid oxidation of dried-salted sardines. Int J Food Sci Technol 25:295-303.
20. Pike A. 2003. Fat characterization. In: Nielsen S, editor. Food Analysis. 3rd ed. New York: Kluwer Academic/Plenum Publishers. p 227-46.
21. Richards M P, Hultin H O. 2000. Effect of pH on Lipid Oxidation Using Trout Hemolysate as a Catalyst: A Possible Role for Deoxyhemoglobin. J Agr Food Chem 48:3141-7.
22. Samaranayaka A G P, Kitts D D, Li-Chan E C Y. 2010. Antioxidative and Angiotensin-1-Converting Enzyme Inhibitory Potential of a Pacific Hake (Merluccius productus) Fish Protein Hydrolysate Subjected to Simulated Gastrointestinal Digestion and Caco-2 Cell Permeation. Journal of Agricultural and Food Chemistry 58:1535-42.
23. Santha N C, Decker E A. 1994. Rapid, sensitive, iron-based spectrophotometric methods for determination of peroxide values of food lipids. Assoc Off Anal Chem Int 77:421-4.
24. Shahidi F. 1997. Natural Antioxidants: Chemistry, Health Effects, and Applications. Champaign, Ill.: AOCS Press.
25. Shi H, Noguchi N, Niki E. 2001. Introducing natural antioxidants. In: Pokorný J, Yanishlieva N, Gordon M, editors. Antioxidants in food: practical application. Cambridge: Woodhead Publishing Limited. p 147-58.
26. Simeonidou S, Govaris A, Vareltzis K. 1997. Quality assessment of seven Mediterranean fish species during storage on ice. Food Res Int 30:479-84.
27. Steinhardt H, Beychok S. 1964. Interaction of protein with hydrogen ions and other small ions and molecules. In: Neurath H, editor. The Proteins. New York: Academic Press. p 139-304.
28. Vermeirssen V, Camp J V, Devos J, Verstraete W. 2003. Release of Angiotensin I Converting Enzyme (ACE) inhibitory activity during in vitro gastrointestinal digestion: from batch experiment to semi-continuous model. Agr Food Chem 51:5680-7.
29. Wang T, Jónsdóttir R, Kristinsson H G, Thorkelsson G, Jacobsen C, Hamaguchi P Y, Ólafsdóttir G. 2010. Inhibition of haemoglobin-mediated lipid oxidation in washed cod muscle and cod protein isolates by Fucus vesiculosus extract and fractions. Food Chem 123:321-30.
30. Wang T, Jónsdóttir R, Ólafsdóttir G. 2009. Total phenolic compounds, radical scavenging and metal chelation of extracts from Icelandic seaweeds. Food Chem 116:240-8.
31. Wolfe K L, Liu R H. 2007. Cellular Antioxidant Activity (CAA) Assay for Assessing Antioxidants, Foods, and Dietary Supplements. Journal of Agricultural and Food Chemistry 55:8896-907.
32. Wu H C, Chen H M, Shiau C Y. 2003. Free amino acids and peptides as related to antioxidant properties in protein hydrolysates of mackerel (Scomber austriasicus). Food Res Int 36:949-57.
33. Xiong Y. 2000. Protein oxidation and implication for muscle food quality. In: Decker E, Faustman C, Lopez-Bote C, editors. Antioxidants in muscle food. New York: John Wiley & Sons, Inc. p 85-111.

1. A process for producing an aquatic protein hydrolysate with enzyme hydrolysis, comprising adding to the protein enzyme mix a natural antioxidant comprising a marine algae extract, before or during the hydrolysis reaction.

2. The process of claim 1, wherein the natural antioxidant comprises an extract comprising a species selected from a Fucus species, an Ascophyllum species, a Laminaria species, an Alaria species, a Palmaria species, a Pelvetia species, a Pyropia species, a Caulerpa species, and a Durvillaea species.

3. The process of claim 1, wherein the natural antioxidant comprises and extract comprising one or more species selected from Fucus spiralis, Fucus vesiculosus, Fucus distichus, Fucus serratus, Fucus ceranoides, Fucus gardneri, Fucus evanescens, Furcellaria lumbricalis, Ascophyllum nodosum, Laminara hyperborea, Laminaria saxatilis, Laminaria saccharina, Laminaria digitata, Laminaria ochroleuca, Laminaria pallida, Laminaria setchellii, Lessonia flavicans, Lessonia nigrescens, Lessonia trabeculata, Lessoniopsis littoralis, Macrocystis intergrifolia, Macrocystis pyrifera, Mastocarpus papillatus, Mastocarpus stellatus, Mazzaella splendens, Monostroma grevillei, Nemacystus decipiens, Nereocystis luetkeana, Osmundea pinnatifida, Palmaria hecatensis, Palmaria mollis, Palmaria palmata, Pelvetia canaliculata, Porphyra purpurea, Porphyra umbilicalis, Postelsia palmaeformis Pterocladia lucida, Pyropia columbina, Pyropia perforata, Pyropia tenera, Pyropia yezoensis, Rissoella verruculosa, Saccharina angustata, Saccharina dentigera, Saccharina groenlandica, Saccharina japonica, Saccharina latissima, Saccharina longicruris, Saccharina sessilis, Sargassum filipendula, Sargassum fusiforme, Sargassum muticum, Stephanocystis osmundacea, Ulva intestinalis, Undaria pinnatifida, Vertebrata lanosa, Alaria esculenta, Alaria marginata, Pelvetia canaliculata, Chondroacanthus canaliculatus, Chondracanthus chamissoi, Chondrus crispus, Caulerpa lentillifera, Caulerpa racemosa, Codium fragile, Costaria costata, Caulerpa sertularioides, Durvillaea antarctica, Durvillaea potatorum, Ecklonia cava, Ecklonia maxima, Ecklonia radiata, Egregia menziesii, Eisenia arborea, Eisenia bicyclis, Eualaria fistulsoa, Eucheuma denticulatum, Gigartina skottsbergii, Gelidiella acerosa, Gelidium corneum, Halopteris scoparia, Iridaea cordata, Jania rubens, and Kappaphycus alvarezii.

4. The process of claim 1, comprising use of an enzyme selected from the group consisting of proteases from marine species, proteases from Bacillus strains, Subtilisin, including Subtilisin from Bacillus licheniformis such as Alcalase® Food Grade, Protamex®, Flavourzyme®, Neutrase®, Protease A “Amano” 2, Protease M “Amano”, Protease P “Amano” 6, Pescalase®, Fromase™, Promod 31™ and Maxatase™.

5. The process of claim 1 or 2, wherein the protein source material is selected from material from fish, including fish muscle, fish skin, fish vicera, fish bones, fish heads, other fish byproducts, and any combination thereof; aquatic mammals; crustaceans, including whole crustaceans, crustacean meat and crustacean shells and process byproducts; and
mollusks.

6. The process of any of claims 1 to 5, comprising grinding or mincing the protein source material in the presence of water, and recovering the minced pulp.

7. The process of claim 6, comprising the steps of:
adjustment of the material prior to the hydrolysis to a protein content in the range of 0.1% to 30% w/v (protein/water);
adjustment of the said material to a pH in the range of 1 to 14;
adjustment of the mixture to a convenient temperature at which the selected enzyme(s) does not become heat inactivated, in the range 0 to 80° C.;
allowing the enzymatic hydrolysis to proceed for a period in the range from about 0.1 to 48 hours or until the degree of hydrolysis (% DH) has reached a desired value in the range 2 to 70% DH; and
stopping the enzymatic hydrolysis by deactivating the enzyme.

8. The process of claim 7, further comprising separating a hydrolyzed aquatic peptide fraction from solid material by concentration, and collecting said fraction.

9. The process of claim 8, further comprising drying said fraction.

10. The process of any of the aforementioned claims, wherein one or more further antioxidant is used in the hydrolysis step, selected from the group consisting of α-Tocopherol, Ascorbic acid, Caffeic acid, Cinnamic acid, Courmaric acid, Carnosic acid, Carnosol, Epicatechin (flavan-3-ol), Ferulic acid, Flavone, Phlorotannins and Rosmarinic acid, and a combination thereof; and preferably selected from alfa-tocopherol, ascorbic acid, and a combination of both.

11. The process of any of the aforementioned claims, comprising stoppage of the enzymatic hydrolysis by deactivation of the employed enzyme with a stoppage step selected from (a) raising the temperature of the said reaction mixture to a level not below 60° C., for 5 to 60 minutes, followed by cooling on ice, and (b) deactivation of the employed enzyme by altering the pH to pH where said enzyme is deactivated, such as a pH below about 5 or above about 8.

12. The process according to any of the aforementioned claims, further comprising subjecting the starting material to protein isolation by extracting the proteins of interest from the starting raw material, and subsequent hydrolysis of the extracted proteins recovered in the process.

13. The process according to any of the aforementioned claims, wherein the said hydrolysis of the said protein source is carried out using gelatin and/or collagen from the said aquatic species as the protein source material.

14. The process according to any of the aforementioned claims, wherein the degree of hydrolysis is followed or measured in the final product.

15. The process according to any of the aforementioned claims, wherein an effective amount of one compound or combinations of two or more compounds that are defined as “natural antioxidants” by a valued scientific prospect such as EFSA and FAO, is used as a further antioxidant agent.

16. The process according to any of the aforementioned claims, wherein separation of the obtained protein hydro lysate is performed by sedimentation.

17. The process according to any of the aforementioned claims, wherein separation of the protein hydrolysate is performed by filtration.

18. The process according to any of the aforementioned claims, wherein separation of the protein hydro lysate is performed by filtration using ultra filtration (UF) membranes, preferably with molecular weight cut-off selected from 30, 10, 5, 3 and 1 kDa.

19. The process according to any of the aforementioned claims, wherein separation of the protein hydrolysate is performed by centrifugation at a speed between 500 and 10000 G and elimination of the residue obtained.

20. The process according to any of the aforementioned claims, wherein recovery of the protein hydrolysate is performed by concentration.

21. The process according to any of claims 1- to 14, wherein recovery of the protein hydrolysate is performed by drying.

22. The process according to any of the aforementioned claims, further comprising incorporation of stabilizers such as antimicrobials to the final product.

23. The process according to any of the aforementioned claims, further comprising deodorization treatment of the final product.

24. An aquatic protein hydrolysate as produced by the process of claim 1, characterized in that in comprises a natural antioxidant selected from the group consisting of marine algae extract such as Fucus vesiculosus extract.

25. A product comprising the protein hydrolysate of claim 19, selected from the group consisting of a food product, a food supplement, pet food, animal feed, fish feed, fertilizer, cosmetic products, pharmaceutical preparations, nutraceutical preparations, and medicaments, characterized in that it comprises an aquatic protein hydrolysate produced by the process of claim 1.

26. The aquatic protein hydrolysate of claim 24, which is in a form selected from capsules; dried form, including powder form, flakes, granules, pellets; liquid, semi-liquid, a suspension, an emulsion, a syrup.

27. The aquatic protein hydrolysate of claim 24, produced from gelatin and/or collagen as protein source material.

28. Use of an aquatic protein hydrolysate of claim 27, for improving gelatin and/or collagen products for incorporation into cosmetics, such as creams, lotions, shampoos, gels, pastes, and masks; food supplements; and foods.

29. Use of an aquatic protein hydrolysate of claim 24, that possesses antioxidant activity, to prevent or treat oxidative stress inside the body and/or on the skin.

30. Use of an aquatic protein hydrolysate of claim 24, that possesses appetite enhancing or suppressing activities.
